(19) United States
(12) Patent Application Publication
(10) Pub. No.: US 2025/0205301 A1
(43) Pub. Date: Jun. 26, 2025

(54) TREATMENT OF UTERINE FIBROIDS

(71) Applicant: Nichole Smith, Los Angeles, CA (US)

(72) Inventor: Nichole Smith, Los Angeles, CA (US)

(21) Appl. No.: 18/848,291

(22) PCT Filed: Mar. 17, 2023
(86) PCT No.: PCT/US2023/015442

§ 371 Date: Sep. 18, 2024

Related U.S. Application Data
(60) Provisional application No. 63/321,155, filed on Mar. 18, 2022.

(51) Int. Cl. A61K 36/8962; A61K 9/00; A61K 9/48; A61K 35/618

(52) U.S. Cl. CPC A61K 36/8962; A61K 9/0053; A61K 9/48; A61K 35/618

(57) ABSTRACT

The present invention relates to the treatment of uterine fibroids, and in particular to the use of a combination of a garlic composition and a mollusk composition to treat or ameliorate uterine fibroids by reducing the size or number of uterine fibroids or to treat or ameliorate one or more symptoms related to uterine fibroids in a subject.

### RELATED APPLICATIONS

The present application claims priority to U.S. Provisional Application No. 63/321,155, filed Mar. 18, 2022, which is incorporated herein by reference in its entirety.

### FIELD OF THE INVENTION

The present invention relates to the treatment of uterine fibroids, and in particular to the use of a combination of a garlic composition and a mollusk composition to treat or ameliorate uterine fibroids by reducing the size or number of uterine fibroids or to treat or ameliorate one or more symptoms related to uterine fibroids in a subject.

### BACKGROUND OF THE INVENTION

Uterine fibroids (also called leiomyomas) are growths made up of the muscle and connective tissue from the wall of the uterus. These growths are usually not cancerous (benign). Fibroids can grow as a single nodule (one growth) or in a cluster. Fibroid clusters can range in size from 1 mm to more than 20 cm (8 inches) in diameter or even larger. These growths can develop within the wall of the uterus, inside the main cavity of the organ or even on the outer surface. Fibroids can vary in size, number and location within and on the uterus.

Approximately 40 to 80% of women have fibroids. While some women do not present symptoms, others commonly do. Common symptoms include excessive or painful bleeding (e.g., bleeding accompanied by cramps or other pain) during menstruation, bleeding between your periods, a feeling of fullness in the lower abdomen and/or bloating, frequent urination, pain during sex, lower back pain, constipation, chronic vaginal discharge, inability to urinate or completely empty the bladder, and increased abdominal distention (enlargement).

In many cases, fibroids are first discovered during a regular exam with a women's health provider. Fibroids can be felt during a pelvic exam and can be found during a gynecologic exam or during prenatal care. Tests for fibroids include ultrasonography, magnetic resonance imaging, computed tomography, hysteroscopy, hysterosalpingography, sonohysterography, and laparoscopy.

Traditional treatment of fibroids depends on several factors: the number of fibroids, the size of the fibroids, location of the fibroids, presentation of symptoms, desire for pregnancy, and desire for uterine preservation. Common treatment option include surgery (e.g., hysterectomy, hysteroscopy, laparoscopy and lapatomy, over-the-counter pain medications, iron supplements. Birth control, gonadotropin-releasing hormone (GnRH) agonists, Elagolix, and Tranexamic acid.

What is needed in the art are new therapies for treating fibroids in women.

### SUMMARY OF THE INVENTION

The present invention relates to the treatment of uterine fibroids, and in particular to the use of a combination of a garlic composition and a mollusk composition to treat or ameliorate uterine fibroids by reducing the size or number of uterine fibroids or to treat or ameliorate one or more symptoms related to uterine fibroids in a subject.

Accordingly, in some preferred embodiments, the present invention provides methods of treating uterine fibroids in a subject suffering therefrom comprising co-administering to the subject an effective amount of a combination of a garlic composition and a mollusk composition under conditions so that the number or size of fibroids in the subject is reduced or one or more symptoms associated with fibroids are treated or ameliorated. In some further preferred embodiments, the present invention provides a combination of a garlic composition and a mollusk composition for use in reducing the number or size of uterine fibroids in a subject or treating or ameliorating one or more symptoms associated with uterine fibroids.

In some preferred embodiments, the one or more symptoms associated with fibroids are selected from the group consisting of excessive or painful bleeding accompanied by cramps or other pain during menstruation, bleeding between your periods, a feeling of fullness in the lower abdomen and/or bloating, frequent urination, pain during sex, lower back pain, constipation, chronic vaginal discharge, inability to urinate or completely empty the bladder, and increased abdominal distention.

In some preferred embodiments, the garlic composition is selected from the group consisting of whole garlic, crushed garlic, a garlic paste, a garlic powder, dried garlic, and a garlic extract.

In some preferred embodiments, the mollusk composition is selected from the group consisting of a whole mollusk, a mollusk paste, a mollusk powder, dried mollusks, and a mollusk extract. In some preferred embodiments, the mollusk is selected from the group consisting or an oyster, a clam and a mussel. In some particularly preferred embodiments, the mollusk is an oyster.

In some preferred embodiments, the combination of a garlic composition and a mollusk composition are provided in a powder or paste. In some preferred embodiments, the combination of a garlic composition and a mollusk composition comprises whole or crushed garlic.

In some preferred embodiments, the combination of a garlic composition and a mollusk composition are administered orally. In some preferred embodiments, the combination of a garlic composition and a mollusk composition are provided in an oral delivery vehicle. In some preferred embodiments, the oral delivery vehicle is selected from the group consisting of a pill and capsule.

In some preferred embodiments, the garlic composition is a powder characterized in having a) a water content of less than 10% by weight (w/w of the powder) and/or b) an allicin content of from 1000 to 20000 micrograms (μg) per gram of powder (μg/g). In some preferred embodiments, the garlic composition has an allicin content of from about 5000 to 15,000 μg/g.

In some preferred embodiments, the mollusk composition is a powder characterized in having one or more the following characteristics: a) a moisture content of less than 8% by weight (w/w of the powder); b) a protein content of from 10% to 90% by weight; c) a taurine content of from 2% to 10% by weight (w/w of the powder); and d) a zinc content of from 100 to 5000 ppm (mg/kg). In some preferred embodiments, the protein content of the mollusk composition is from 30% to 75% by weight, and most preferably from 50% to 70% by weight (w/w of the powder). In some preferred embodiments, the zinc content of the mollusk composition is from 500 to 2500 ppm (mg/kg).

In some preferred embodiments, the present invention provides an oral delivery vehicle comprising a combination of from 10 to 1000 mg of a garlic composition and from 10 to 1000 mg of a mollusk composition. In some preferred embodiments, the garlic composition is a powder and is characterized in having a) a water content of less than 10% by weight (w/w of the powder) and/or b) an allicin content of from 1000 to 20000 micrograms (μg) per gram of powder (μg/g). In some preferred embodiments, the garlic composition has an allicin content of from about 5000 to 15,000 μg/g. In some preferred embodiments, the mollusk composition is a powder and is characterized in having one or more the following characteristics: a) a moisture content of less than 8% by weight (w/w of the powder); b) a protein content of from 10% to 90% by weight; c) a taurine content of from 2% to 10% by weight (w/w of the powder); and d) a zinc content of from 100 to 5000 ppm (mg/kg). In some preferred embodiments, the protein content of the mollusk composition is from 30% to 75% by weight, and most preferably from 50% to 70% by weight (w/w of the powder). In some preferred embodiments, the zinc content of the mollusk composition is from 500 to 2500 ppm (mg/kg). In some preferred embodiments, the oral delivery vehicle is provided for use in reducing the number or size of uterine fibroids in a subject or treating or ameliorating one or more symptoms associated with uterine fibroids. In some preferred embodiments, the one or more symptoms associated with fibroids are selected from the group consisting of excessive or painful bleeding accompanied by cramps or other pain during menstruation, bleeding between your periods, a feeling of fullness in the lower abdomen and/or bloating, frequent urination, pain during sex, lower back pain, constipation, chronic vaginal discharge, inability to urinate or completely empty the bladder, and increased abdominal distention.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the technology may be readily combined, without departing from the scope or spirit of the technology. In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, “active” or “activity” refers to native or naturally occurring biological and/or immunological activity.

As used herein, the term “individual” refers to vertebrates, particularly members of the mammalian species. The term includes but is not limited to domestic animals, sports animals, primates, and humans.

As used herein, the term “effective amount” refers to the amount of a composition sufficient to effect beneficial or desired results. An effective amount can be administered in one or more administrations, applications, or dosages and is not intended to be limited to a particular formulation or administration route.

As used herein, the term “administration” refers to the act of giving a drug, prodrug, or other agent, or therapeutic treatment to a subject. Exemplary routes of administration to the human body can be through the eyes (ophthalmic), mouth (oral), skin (transdermal, topical), nose (nasal), lungs (inhalant), oral mucosa (buccal), ear, by injection (e.g., intravenously, subcutaneously, intratumorally, intraperitoneally, etc.), and the like.

As used herein, the term “co-administration” refers to the administration of at least two agents or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent.

As used herein, the term “pharmaceutical composition” refers to the combination of an active agent with a carrier, inert or active, making the composition especially suitable for therapeutic use.

The terms “pharmaceutically acceptable” or “pharmacologically acceptable”, as used herein, refer to compositions that do not substantially produce adverse reactions, e.g., toxic, allergic, or immunological reactions, when administered to a subject.

As used herein, the term “treating” includes reducing or alleviating at least one adverse effect or symptom of a disease or disorder through introducing in any way a therapeutic composition of the present technology into or onto the body of a subject. “Treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

As used herein, the term “garlic composition” refers to a composition prepared from garlic and includes whole garlic compositions such as a garlic paste, crushed garlic or freeze-dried garlic, extracts from garlic (e.g., solvent extracts of garlic) and purified components from garlic. The garlic compositions preferably comprise allicin (allyl thiosulfinate).

As used herein, the term “mollusk composition” refers to a composition prepared from a mollusk and includes whole mollusk compositions such as a mollusk paste or freeze-dried mollusk powder, extracts from mollusks (e.g., solvent extracts of garlic) and purified components from mollusks.

### DETAILED DESCRIPTION OF THE INVENTION

The present invention relates to the treatment of uterine fibroids, and in particular to the use of a combination of a garlic composition and a mollusk composition to treat or ameliorate uterine fibroids by reducing the size or number of uterine fibroids or to treat or ameliorate one or more symptoms related to uterine fibroids in a subject.

In some embodiments, the present invention provides methods of treating uterine fibroids in a subject suffering therefrom comprising co-administering to the subject an effective amount of a combination of a garlic composition and a mollusk composition under conditions so that the number or size of fibroids in the subject is reduced or one or more symptoms associated with fibroids are treated or ameliorated. In some preferred embodiments, the present invention provides a combination of a garlic composition and a mollusk composition for use in reducing the number or size of fibroids in a subject in need thereof or to treat or ameliorate symptoms associated with fibroids in a subject in need thereof. In some preferred embodiments, the symptoms are one or more of excessive or painful bleeding during menstruation, such as bleeding accompanied by cramps or other pain, bleeding between periods, a feeling of fullness in the lower abdomen and/or bloating, frequent urination, pain during sex, lower back pain, constipation, chronic vaginal discharge, inability to urinate or completely empty the bladder, and increased abdominal distention.

In some preferred embodiments, the garlic composition is selected from the group consisting of whole garlic, a garlic paste, crushed garlic, a garlic powder, and a garlic extract. In some preferred embodiments, the garlic composition is an air-dried or freeze-dried powder prepared from whole garlic or a garlic extract. In some preferred embodiments, the garlic powder is characterized in having one or both the following characteristics: a) a water content of less than 10% by weight (w/w of the powder) and/or b) an allicin content of from 1000 to 20000 micrograms (μg) per gram of powder (μg/g), most preferably from about 5000 to 15,000 μg/g.

In some preferred embodiments, the mollusk composition is selected from the group consisting of a whole mollusk, a mollusk paste, a mollusk powder, and a mollusk extract. The present invention is not limited to the use of any particular mollusk. In some preferred embodiments, the mollusk is an oyster (Crassostrea gigas), claim or mussel (e.g., green lipped mussel). In some particularly preferred embodiments, the mollusk is an oyster. In some preferred embodiments, a whole mollusk is utilized. Suitable forms of whole mollusks include, but are not limited to, fresh, raw mollusks, canned mollusks, boiled mollusks, cooked mollusks, smoked mollusks, and whole dried or freeze-dried mollusks and the like. In other preferred embodiments, the mollusk composition is a mollusk paste. In some preferred embodiments, the mollusk paste may be prepared from fresh, raw mollusks, canned mollusks, boiled mollusks, cooked mollusks, or smoked mollusks. In other preferred embodiments, the mollusk composition is a mollusk powder. In some preferred embodiments, the mollusk powder is prepared from dried or freeze-dried mollusks. In still other preferred embodiments, the mollusk composition is a mollusk extract. In some embodiments, the mollusk extract is a hydrolysate or solvent extract from mollusks. Mollusks extracts and powders are commercially available, for example, Good Health Oyster Plus™ (New Zealand), Horbaach Green Lipped Mussel capsules (powder, New Zealand) and described in U.S. Appls. 20040043080, 20040185166, and 20010041212, all of which are incorporated herein by reference in their entirety. In some preferred embodiments, the mollusks extracts or powders, and in particularly preferred embodiments oyster extracts and powders, comprise from 1 to 20% zinc by weight in addition to taurine and/or mollusk peptides and proteins.

In some preferred embodiments, a mollusk powder (e.g., a freeze dried oyster or oyster extract powder) composition of the present invention is characterized in having one or more the following characteristics: a) a moisture content of less than 8% by weight (w/w of the powder), b) a protein content of from 10% to 90% by weight, and more preferably from 30% to 75% by weight, and most preferably from 50% to 70% by weight (w/w of the powder), c) a taurine content of from 2% to 10% by weight (w/w of the powder) and d) a zinc content of from 100 to 5000 ppm (mg/kg), most preferably from 500 to 2500 ppm (mg/kg). In some embodiments, the powder has one of characteristics a, b, c, and d. In some embodiments, the powder has at least two of characteristics a, b, c, and d. In some embodiments, the powder has two of characteristics a, b, c, and d. In some embodiments, the powder has at least three of characteristics a, b, c, and d. In some embodiments, the powder has three of characteristics a, b, c, and d. In some embodiments, the powder has all of characteristics a, b, c, and d. In some embodiments, the powder has characteristics a and b. In some embodiments, the powder has characteristics a and c. In some embodiments, the powder has characteristics a and d. In some embodiments, the powder has characteristics b and c. In some embodiments, the powder has characteristics b and d. In some embodiments, the powder has characteristics a, b and c. In some embodiments, the powder has characteristics a, b and d. In some embodiments, the powder has characteristics b, c, and d.

In some preferred embodiments, the compositions and formulations of the present invention have a content of at least 1%, 2%, 5%, 10%, 20%, 30%, 40% or 50% of a garlic composition on a w/w basis (total weight of garlic composition/total weight of the formulation) and at least 1%, 2%, 5%, 10%, 20%, 30%, 40% or 50% of a mollusk composition on a w/w basis (total weight of mollusk composition/total weight of the formulation), or from about 1% to 50%, 2% to 40%, 5% to 40%, 10% to 40%, 1% to 30%, 2% to 30%, 5% to 30%, 10% to 30%, 1% to 25%, 2% to 25%, 5% to 25%, or 10% to 25% garlic composition on w/w basis and from about 1% to 50%, 2% to 40%, 5% to 40%, 10% to 40%, 1% to 30%, 2% to 30%, 5% to 30%, 10% to 30%, 1% to 25%, 2% to 25%, 5% to 25%, or 10% to 25% mollusk composition on w/w basis.

In some embodiments, the formulations are suitable for human consumption on a daily basis for an extended period of time, e.g., 2 weeks, 1 month, 2 months, 6 months, 1 year or 2 years, when provided in daily dosage of from 100 mg to 100 grams. In some preferred embodiments, an effective amount of a combination of a garlic composition and an mollusk composition is from about 10 milligrams to 20 grams of the garlic composition and from 10 milligrams to 100 grams of the mollusk composition. In some preferred embodiments, an effective amount of a combination of a garlic composition and an mollusk composition is from about 100 milligrams to 20 grams of the garlic composition and from 100 milligrams to 100 grams of the mollusk composition.

In some preferred embodiments, garlic and oyster powders or extracts are utilized. In these embodiments, an effective amount of a combination of a garlic composition and an oyster composition is from about 10 milligrams to 1 gram of the garlic powder or extract and from 10 milligrams to 2 grams of the mollusk garlic powder or extract. In some preferred embodiments, an effective amount of a combination of a garlic powder or extract and a mollusk powder or extract is from about 100 milligrams to 1 gram of the garlic powder or extract and from 100 milligrams to 2 grams of the mollusk powder or extract.

Suitable 500 mg capsule formulations of a combination of a commercially available garlic extract powder comprising 9,000 μg/g allicin (from Pharmax Freeze-Dried Garlic Caps) and a commercially available oyster powder having a protein content of about 52.7% w/w, a moisture content of 4.6% w/w, a taurine content of 5.5% (Healthlife Oyster Meat Freeze-dried Powder) or a commercially available oyster extract powder (Oyster Power Zinc Rich Supplement) having a moisture content of 1.2% w/w and a zinc content of 1692 mg/kg are described in the following Table. From 1 to 20 capsules, and preferably from 2 to 10 of the capsules may be consumed daily by an average adult female.

| | Free-dried garlic | Freeze-dried oyster | Oyster extract |
|---|---|---|---|
| | powder (mg per | powder (mg per | powder (mg per |
| Formulation | 500 mg capsule) | 500 mg capsule) | 500 mg capsule) |
| 1 | 100 | 400 | — |
| 2 | 200 | 300 | — |
| 3 | 300 | 200 | — |
| 4 | 400 | 100 | — |
| 5 | 100 | — | 400 |
| 6 | 200 | — | 300 |
| 7 | 300 | — | 200 |
| 8 | 400 | — | 100 |
| 9 | 250 | 250 | — |
| 10 | 250 | — | 250 |

In some embodiments, the formulations further comprise a food safe antioxidant. In some preferred embodiments, the food safe antioxidant does not occur naturally in garlic or mollusks.

In some embodiments, the formulations comprise an excipient or carrier. In some preferred embodiments, the excipient or carrier does not naturally occur in garlic or mollusks.

In some embodiments, the formulations are provided in an oral delivery vehicle, food product, nutritional supplement, dietary supplement or functional food.

Provided herein are pharmaceutical formulations comprising a therapeutically effective amount of the garlic/mollusk compositions according to the present technology and a pharmaceutically acceptable carrier, diluent, or excipient (including combinations thereof).

A preferred formulation according to the technology comprises or consists of a therapeutically effective amount of the garlic and mollusk compositions. In some embodiments, it includes a pharmaceutically acceptable carrier, diluent, or excipient (including combinations thereof). Acceptable carriers or diluents for therapeutic use are well known in the pharmaceutical art and are described, for example, in Remington's Pharmaceutical Sciences, Mack Publishing Co. (A. R. Gennaro edit. 1985). The choice of pharmaceutical carrier, excipient, or diluent is selected with regard to the intended route of administration and standard pharmaceutical practice. The pharmaceutical comprise as, or in addition to, the carrier, excipient, or diluent any suitable binder(s), lubricant(s), suspending agent(s), coating agent(s), and/or solubilizing agent(s).

This pharmaceutical composition will desirably be provided in a sterile form. It may be provided in unit dosage form and will generally be provided in a sealed container. A plurality of unit dosage forms may be provided.

Pharmaceutical compositions within the scope of the present technology may include one or more of the following: preserving agents, solubilizing agents, stabilizing agents, wetting agents, emulsifiers, sweeteners, colorants, flavoring agents, odorants, and/or salts. Compounds of the present technology may themselves be provided in the form of a pharmaceutically acceptable salt. In addition, embodiments may comprise buffers, coating agents, antioxidants, suspending agents, adjuvants, excipients, and/or diluents. Examples of preservatives include sodium benzoate, sorbic acid, and esters of p-hydroxybenzoic acid. In some preferred embodiments, preserving agents, solubilizing agents, stabilizing agents, wetting agents, emulsifiers, sweeteners, colorants, flavoring agents, odorants, and/or salts do not occur naturally in garlic or mollusks.

They may also contain other therapeutically active agents in addition to compounds of the present technology. Where two or more therapeutic agents are used they may be administered separately (e.g., at different times and/or via different routes) and therefore do not always need to be present in a single composition. Thus, combination therapy is within the scope of the present technology.

The routes for administration (delivery) include, but are not limited to, one or more of: oral (e.g. as a tablet, capsule, or as an ingestible solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, via the penis, vaginal, epidural, sublingual.

It is to be understood that not all of the agent need be administered by the same route. Likewise, if the composition comprises more than one active component, then those components may be administered by different routes.

In some embodiments, pharmaceutical compositions adapted for oral administration are provided as capsules or tablets; as powders or granules; as solutions, syrups or suspensions (in aqueous or non-aqueous liquids); as edible foams or whips; or as emulsions. Tablets or hard gelatin capsules may comprise lactose, maize starch or derivatives thereof, stearic acid or salts thereof. Soft gelatin capsules may comprise vegetable oils, waxes, fats, semi-solid, or liquid polyols etc. Solutions and syrups may comprise water, polyols and sugars. For the preparation of suspensions, oils (e.g., vegetable oils) may be used to provide oil-in-water or water-in-oil suspensions. An active agent intended for oral administration may be coated with or admixed with a material that delays disintegration and/or absorption of the active agent in the gastrointestinal tract (e.g., glyceryl monostearate or glyceryl distearate may be used). Thus, the sustained release of an active agent may be achieved over many hours and, if necessary, the active agent can be protected from being degraded within the stomach. Pharmaceutical compositions for oral administration may be formulated to facilitate release of an active agent at a particular gastrointestinal location due to specific pH or enzymatic conditions. Alternatively, the agent of the present technology can be administered in the form of a suppository or pessary, or it may be applied topically in the form of a gel, hydrogel, lotion, solution, cream, ointment or dusting powder. The agent of the present technology may also be dermally or transdermally administered, for example, by the use of a skin patch. They may also be administered by the pulmonary or rectal routes. They may also be administered by the ocular route. For ophthalmic use, the compounds can be formulated as micronized suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

The specific dose level and frequency of dosage for any particular patient may be varied and will depend upon a variety of factors including the activity of the specific compound employed; the metabolic stability and length of action of that compound; the age, body weight, general health, sex, diet, mode and time of administration; rate of excretion; drug combination; the severity of the particular condition; and the individual undergoing therapy. The agent and/or the pharmaceutical composition of the present technology may be administered in accordance with a regimen of from 1 to 10 times per day, such as once or twice per day. For oral and parenteral administration to human patients, the daily dosage level of the agent may be in single or divided doses.

Depending upon the need, the agent may be administered at a dose of from 0.01 to 30 mg/kg body weight, such as from 0.1 to 10 mg/kg or from 0.1 to 1 mg/kg body weight. Naturally, the dosages mentioned herein are exemplary of the average case. There can, of course, be individual instances where higher or lower dosage ranges are merited.

### EXPERIMENTAL

### Example 1

A combination of a garlic extract and oysters was orally administered to two subjects diagnosed with uterine fibroids and experiencing symptoms including heavy menstrual bleeding, menstrual cramps, and bleeding outside menstruation. The garlic extract was prepared by crushing or dicing two garlic cloves (approximately 0.3 oz) and mixing with 16 oz. water. 16 oz. of the garlic extract was consumed over the course of each 24 hour period. For subject 1, for the first three months, 2.2 oz. oysters (in oil, drained and rinsed) were consumed daily, followed by 1.3 oz. daily after three months continuously for 1 year. Subject 2 has been treated for six months with the initial dosages for Subject 1 and then reduced to the lower dose after six months. Subject 1 and 2 both observed reduced menstrual bleeding, blood clots, and cramping associated with bleeding with 48 hours of beginning treatment. Subject 1 and 2 also observed reduced bleeding outside of menstruation. Removal of the treatment resulted in a return of symptoms but symptoms did not return to pre-treatment levels of severity for several months. After 1 year, Subject 1 and 2 used the second dosage starting 3 days before their menstrual cycle and ending 3 days after the cycle for continued symptoms management. Both subjects observed that neither garlic only nor oysters only produced the same effect as the combination of oysters and garlic.

### Example 2

Capsules containing a garlic composition and oyster composition were prepared as follows. An oven was set to 170 degrees. Six canned oysters were drained, rinsed and weighed (1.283 oz). The oysters were dried in the oven for 7 hours. The dried weight was 0.329 oz. The dried oysters were ground to a powder. Two garlic cloves were then mashed with a mortar and pestle for 20 seconds and then left to sit for 2 minutes. 24 drops of water were initially added to the mashed garlic, followed by additional 20 drops. Capsules were filled with a mixture of oyster powder and three drops of garlic extract. The capsules were then frozen for storage.

### Example 3 (Prophetic)

500 mg capsules are prepared by combining a commercially available garlic extract powder comprising 9,000 μg/g allicin (from Pharmax Freeze-Dried Garlic Caps) and either 1) a commercially available oyster powder having a protein content of about 52.7% w/w, a moisture content of 4.6% w/w, a taurine content of 5.5% (Healthlife Oyster Meat Freeze-dried Powder) or 2) a commercially available oyster extract powder (Oyster Power Zinc Rich Supplement) having a moisture content of 1.2% w/w and a zinc content of 1692 mg/kg. The powders in the amounts designated in the following table are mixed and filled into a two-piece gelatin capsule.

| | Free-dried garlic | Freeze-dried oyster | Oyster extract |
|---|---|---|---|
| | powder (mg per | powder (mg per | powder (mg per |
| Formulation | 500 mg capsule) | 500 mg capsule) | 500 mg capsule) |
| 1 | 100 | 400 | — |
| 2 | 200 | 300 | — |
| 3 | 300 | 200 | — |
| 4 | 400 | 100 | — |
| 5 | 100 | — | 400 |
| 6 | 200 | — | 300 |
| 7 | 300 | — | 200 |
| 8 | 400 | — | 100 |
| 9 | 250 | 250 | — |
| 10 | 250 | — | 250 |

### Example 4 (Prophetic)

One part garlic juice from crushed garlic is combined with one part oyster powder and one or more flavoring agents. The resulting paste is refrigerated. For oral ingestion, the paste is mixed with water to form a beverage and consumed by the subject by drinking.

### Example 5 (Prophetic)

Oysters are prepared with one or more flavoring agents are ingested once or twice daily and a pill or concentrated liquid containing active allicin is ingested every 4 hours daily.

1. A method of treating uterine fibroids in a subject suffering therefrom comprising co-administering to the subject an effective amount of a combination of a garlic composition and a mollusk composition under conditions so that the number or size of fibroids in the subject is reduced or one or more symptoms associated with fibroids are treated or ameliorated.

2. The method of claim 1, wherein the one or more symptoms associated with fibroids are selected from the group consisting of excessive or painful bleeding accompanied by cramps or other pain during menstruation, bleeding between your periods, a feeling of fullness in the lower abdomen and/or bloating, frequent urination, pain during sex, lower back pain, constipation, chronic vaginal discharge, inability to urinate or completely empty the bladder, and increased abdominal distention.

3. The method of any one of claims 1 to 2, wherein the garlic composition is selected from the group consisting of whole garlic, crushed garlic, a garlic paste, a garlic powder, dried garlic, and a garlic extract.

4. The method of any one of claims 1 to 3, wherein the mollusk composition is selected from the group consisting of a whole mollusk, a mollusk paste, a mollusk powder, dried mollusks, and a mollusk extract.

5. The method of claim 4, wherein the mollusk is selected from the group consisting or an oyster, a clam and a mussel.

6. The method of claim 4, wherein the mollusk is an oyster.

7. The method of any one of claims 1 to 6, wherein the combination of a garlic composition and a mollusk composition are administered orally.

8. The method of any one of claims 1 to 7, wherein the combination of a garlic composition and a mollusk composition are provided in a powder or paste.

9. The method of any one of claims 1 to 7, wherein the combination of a garlic composition and a mollusk composition comprises whole or crushed garlic.

10. The method of any one of claims 1 to 9, wherein the combination of a garlic composition and a mollusk composition are provided in an oral delivery vehicle.

11. The method of claim 10, wherein the oral delivery vehicle is selected from the group consisting of a pill and capsule.

12. The method of any one of claims 1 to 11, wherein the garlic composition is a powder characterized in having a) a water content of less than 10% by weight (w/w of the powder) and/or b) an allicin content of from 1000 to 20000 micrograms (μg) per gram of powder (μg/g).

13. The method of claim 12, wherein the garlic composition has an allicin content of from about 5000 to 15,000 μg/g.

14. The method of any one of claims 1 to 13, wherein the mollusk composition is a powder characterized in having one or more the following characteristics:
a) a moisture content of less than 8% by weight (w/w of the powder);
b) a protein content of from 10% to 90% by weight;
c) a taurine content of from 2% to 10% by weight (w/w of the powder); and
d) a zinc content of from 100 to 5000 ppm (mg/kg).

15. The method of claim 14, wherein the protein content of the mollusk composition is from 30% to 75% by weight, and most preferably from 50% to 70% by weight (w/w of the powder).

16. The method of claim 14, wherein the zinc content of the mollusk composition is from 500 to 2500 ppm (mg/kg).

17. An oral delivery vehicle comprising a combination of from 10 to 1000 mg of a garlic composition and from 10 to 1000 mg of a mollusk composition.

18. Oral delivery vehicle of claim 17, wherein the garlic composition is a powder and is characterized in having a) a water content of less than 10% by weight (w/w of the powder) and/or b) an allicin content of from 1000 to 20000 micrograms (μg) per gram of powder (μg/g).

19. Oral delivery vehicle of claim 18, wherein the garlic composition has an allicin content of from about 5000 to 15,000 μg/g.

20. Oral delivery vehicle of any one of claims 17 to 18, wherein the mollusk composition is a powder and is characterized in having one or more the following characteristics:
a) a moisture content of less than 8% by weight (w/w of the powder);
b) a protein content of from 10% to 90% by weight;
c) a taurine content of from 2% to 10% by weight (w/w of the powder); and
d) a zinc content of from 100 to 5000 ppm (mg/kg).

21. Oral delivery vehicle of claim 20, wherein the protein content of the mollusk composition is from 30% to 75% by weight, and most preferably from 50% to 70% by weight (w/w of the powder).

22. Oral delivery vehicle of claim 20, wherein the zinc content of the mollusk composition is from 500 to 2500 ppm (mg/kg).

23. Oral delivery vehicle of any one of claims 17 to 22 for use in reducing the number or size of uterine fibroids in a subject or treating or ameliorating one or more symptoms associated with uterine fibroids.

24. Use of claim 23, wherein the one or more symptoms associated with fibroids are selected from the group consisting of excessive or painful bleeding accompanied by cramps or other pain during menstruation, bleeding between your periods, a feeling of fullness in the lower abdomen and/or bloating, frequent urination, pain during sex, lower back pain, constipation, chronic vaginal discharge, inability to urinate or completely empty the bladder, and increased abdominal distention.

25. Combination of a garlic composition and a mollusk composition for use in reducing the number or size of uterine fibroids in a subject or treating or ameliorating one or more symptoms associated with uterine fibroids.

26. Use of claim 25, wherein the one or more symptoms associated with fibroids are selected from the group consisting of excessive or painful bleeding accompanied by cramps or other pain during menstruation, bleeding between your periods, a feeling of fullness in the lower abdomen and/or bloating, frequent urination, pain during sex, lower back pain, constipation, chronic vaginal discharge, inability to urinate or completely empty the bladder, and increased abdominal distention.

27. Use of any one of claims 25 to 26, wherein the garlic composition is selected from the group consisting of whole garlic, crushed garlic, a garlic paste, a garlic powder, dried garlic, and a garlic extract.

28. Use of any one of claims 25 to 27, wherein the mollusk composition is selected from the group consisting of a whole mollusk, a mollusk paste, a mollusk powder, dried mollusk, and a mollusk extract.

29. Use of claim 28, wherein the mollusk is selected from the group consisting of an oyster, a clam and a mussel.

30. Use of claim 29, wherein the mollusk is an oyster.

31. Use of any one of claims 25 to 30, wherein the combination of a garlic composition and a mollusk composition are administered orally.

32. Use of any one of claims 25 to 31, wherein the combination of a garlic composition and a mollusk composition are provided in a powder.

33. Use of any one of claims 25 to 31, wherein the combination of a garlic composition and a mollusk composition comprises whole or crushed garlic.

34. Use of any one of claims 25 to 33, wherein the combination of a garlic composition and a mollusk composition are provided in an oral delivery vehicle.

35. Use of claim 34, wherein the oral delivery vehicle is selected from the group consisting of a pill and capsule.

36. Use of any one of claims 25 to 35, wherein the garlic composition is characterized in having a) a water content of less than 10% by weight (w/w of the powder) and/or b) an allicin content of from 1000 to 20000 micrograms (μg) per gram of powder (μg/g).

37. Use of claim 36, wherein the garlic composition has an allicin content of from about 5000 to 15,000 μg/g.

38. Use of any one of claims 25 to 37, wherein the mollusk composition is characterized in having one or more the following characteristics:
a) a moisture content of less than 8% by weight (w/w of the powder);
b) a protein content of from 10% to 90% by weight;
c) a taurine content of from 2% to 10% by weight (w/w of the powder); and
d) a zinc content of from 100 to 5000 ppm (mg/kg).

39. The method of claim 38, wherein the protein content of the mollusk composition is from 30% to 75% by weight, and most preferably from 50% to 70% by weight (w/w of the powder).

40. The method of claim 38, wherein the zinc content of the mollusk composition is from 500 to 2500 ppm (mg/kg).
